(19) United States
(12) Patent Application Publication
(10) Pub. No.: US 2021/0202086 A1
(43) Pub. Date: Jul. 1, 2021

(54) SYSTEM, METHOD, AND APPARATUS FOR COLLECTING AND ANALYZING PHYSIOLOGIC, MEDICAL, AND PSYCHOMETRIC DATA IN SUPPORT OF CLINICAL DECISION MAKING

(71) Applicant: eCare21, Inc., Tampa, FL (US)

(72) Inventor: Vadim Cherdak, Tampla, FL (US)

(21) Appl. No.: 17/122,558

(22) Filed: Dec. 15, 2020

Related U.S. Application Data
(60) Provisional application No. 62/948,279, filed on Dec. 15, 2019.

(51) Int. Cl. G16H 40/67; G16H 40/63; G16H 10/60; G16H 50/30; G06N 20/00

(52) U.S. Cl. CPC G16H 40/67; G16H 40/63; G06N 20/00; G16H 50/30; G16H 10/60

(57) ABSTRACT

Systems, methods, and apparatuses for remote patient monitoring (RPM) and/or care management of patients outside of a medical facility are provided. The systems, methods, and apparatuses can act as a middleware-type connection between instruments used by patients, medical facility records management systems, and healthcare provider record management systems. The systems, methods, and apparatuses can accept data in any applicable format from instruments used by a patient outside the medical facility. The data can be standardized, integrated based on time, relevancy, and reliability, and provided to a database. The data can be provided to a dashboard for monitoring, allowing healthcare providers to monitor the levels of patients remotely with relative ease, even when the patients are not in a medical facility.

### CROSS REFERENCE TO RELATED APPLICATION

This application claims the benefit of U.S. Provisional Application Ser. No. 62/948,279, filed Dec. 15, 2019, which is incorporated herein by reference in its entirety.

### FIELD OF INVENTION

Embodiments of the subject invention provide systems, methods, and apparatuses for commercial use of remote patient monitoring (RPM) and care management of patients outside of a medical facility. The systems, methods, and apparatuses support clinical decision-making by: collecting physiologic, medical, and psychometric data during the times when patients are outside of medical facilities; analyzing the data against smart contracts, medical data analytics, and historical data; and propagating actionable intelligence. The actionable intelligence can be propagated to chronic care management systems (CCM) and/or electronic medical records management systems (ERMS), and delivered to family caregivers, homecare providers, care managers, hospital staff, and/or physicians in real time to improve results, decision making, and treatment protocols.

### BACKGROUND

There is currently an epidemic of patients requiring high-risk therapeutic treatment, the main causes of which are a consequence of aging (e.g., type II diabetes mellitus, hypertension, vascular disease). The number of patients requiring high-risk therapeutic treatment is increasing by 10% per year in most of the world's developed countries, with patients over 65 years old accounting for most of this increase. The world's healthcare systems are buckling under the weight of the increased cost and demand for services precipitated by the 10,000 Baby Boomers turning 65 each day.

Additionally, the majority of health problems happen outside of medical healthcare facilities, while the majority of data collected is limited to only that which is collected within medical healthcare facilities. At best, modern healthcare is reactive. Without question, healthcare decisions are based on a very narrow slice of a patient's physiologic, medical, and psychological makeup. This results in healthcare providers making decisions and formulating healthcare treatment protocols by extrapolating out from a small foundation (or sample size) of clinical data. To ensure positive results practitioners overprescribe, over-treat, and over-expand the scope of necessary care. Additionally, by requiring patients to come to the physician, patients are exposed to the germs and bacteria of other patients, increasing risk of disease. The result is less than effective preventive care from both a management of scarce resources perspective as well as an ability to ensure maximum results at minimum risk, coupled with an inability to manage health risk factors and promote health and wellness habits.

### BRIEF SUMMARY

In view of the above, there is a need in the art for expanded in-home care services coupled with efficient provisioning of healthcare services. There is also a need for systems and methods for collecting physiologic, medical, and/or psychometric data during the times when patients are outside of medical facilities, as well as integrating this data into the data stores of medical facilities. There is also a need for systems and methods that analyze the data against smart contracts, medical data analytics, and/or historical data to create actionable intelligence that can be promulgated to the person or persons (e.g., medical health professionals, such as physicians, physician assistants, nurse practitioners, etc.) best able to act on the information. Such an increased purview into the complete physiologic, medical, and/or psychological status of a patient over long periods of time can allow healthcare providers to apply the rigors and methodologies of longitudinal studies to every patient's treatment protocol. Promulgating actionable intelligence improves care, minimizes adverse effects of chronic conditions, medical treatments, and post-operative care, and allows healthcare providers to track their own activities in order to ensure compliance and seek reimbursement from group healthcare plans.

Embodiments of the subject invention provide systems, methods, and apparatuses for remote patient monitoring (RPM) and/or care management of patients outside of a medical facility. The systems, methods, and apparatuses can act as a middleware-type connection between instruments used by patients, medical facility (e.g., hospital) records management systems, and healthcare provider (e.g., physician, physician assistant, nurse practitioner, etc.)

record management systems. The systems, methods, and apparatuses can accept data in any applicable format from instruments (e.g., sphygmomanometer, blood glucose monitor, thermometer, etc.) used by a patient outside the medical facility. The data can be standardized, integrated based on time, relevancy, and reliability (e.g., based on the type of device from which it comes and the historical reliability of such devices), and provided to a database. The standardized, integrated data can be provided to a dashboard and linked with records management system(s) of any number of medical facilities and/or healthcare providers for monitoring. This allows healthcare providers to monitor the levels (e.g., blood glucose level of a diabetes patient) of patients remotely with relative ease, even when the patients are not in a medical facility.

In an embodiment, a system for remote monitoring of at least one medical patient can comprise: a processor; a first display; and a machine-readable medium in operable communication with the processor, the machine-readable medium having instructions stored thereon that, when executed by the processor, perform the following steps: collecting RPM data from at least one RPM device in use by the at least one medical patient outside of a medical facility, the collected RPM data comprising measurement values of the at least one RPM device; standardizing the collected RPM data; categorizing the standardized RPM data based on a time the RPM data was collected from the at least RPM device and the type of RPM device from which the RPM data was collected; storing the categorized RPM data in a database; and organizing the categorized RPM data in a dashboard in a graphical user interface (GUI) displayed on the first display where it is monitored by a medical professional and used as actionable intelligence in providing medical care to the at least one medical patient.

In another embodiment, a method for remote monitoring of at least one medical patient can comprise: collecting RPM data from at least one RPM device in use by the at least one medical patient outside of a medical facility, the collected RPM data comprising measurement values of the at least one RPM device; standardizing the collected RPM data; categorizing the standardized RPM data based on a time the RPM data was collected from the at least RPM device and the type of RPM device from which the RPM data was collected; storing the categorized RPM data in a database; and organizing the categorized RPM data in a dashboard in a GUI displayed on a first display where it is monitored by a medical professional and used as actionable intelligence in providing medical care to the at least one medical patient.

### BRIEF DESCRIPTION OF DRAWINGS

FIG. 1 shows a schematic view of a system for remote patient monitoring and chronic care management, depicting general stages associated therewith, according to an embodiment of the subject invention.

FIG. 2 shows a schematic view of an intelligent monitoring environment (IME) and decision support engine (DSE), depicting general stages associated therewith, depicting general stages associated therewith, according to an embodiment of the subject invention.

FIG. 3 shows a virtual triage dashboard for medical practitioners to evaluate patients and respond to warnings and suggestions generated by the DSE based on remote patient monitoring (RPM) data, according to an embodiment of the subject invention.

FIG. 4 shows a sample graphic report of a warning generated based on a patient's out-of-norm condition(s), according to an embodiment of the subject invention.

FIG. 5 shows an image of sample graphic tools for medical personnel to assess a patient's out-of-norm condition(s), according to an embodiment of the subject invention.

FIG. 6 shows an image of sample graphic tools for medical personnel to generate a rules-based method for remote patient care and treatment, according to an embodiment of the subject invention.

FIG. 7 shows a formula for estimating relative risk of a patient (e.g., an elderly patient) being hospitalized during a 6-month period, according to an embodiment of the subject invention.

FIG. 8 shows a formula for developing a parametric model, according to an embodiment of the subject invention.

FIG. 9 shows a table listing some of the many features and functionality of embodiments of the subject invention.

### DETAILED DESCRIPTION

Embodiments of the subject invention provide systems, methods, and apparatuses for remote patient monitoring (RPM) and/or care management of patients outside of a medical facility. The systems, methods, and apparatuses can act as a middleware-type connection between instruments used by patients, medical facility (e.g., hospital) records management systems, and healthcare provider (e.g., physician, physician assistant, nurse practitioner, etc.) record management systems. The systems, methods, and apparatuses can accept data in any applicable format from instruments (e.g., sphygmomanometer, blood glucose monitor, thermometer, etc.) used by a patient outside the medical facility. The data can be standardized, integrated based on time, relevancy, and reliability (e.g., based on the type of device from which it comes and the historical reliability of such devices), and provided to a database. The standardized, integrated data can be provided to a dashboard and linked with records management system(s) of any number of medical facilities and/or healthcare providers for monitoring. This allows healthcare providers to monitor the levels (e.g., blood glucose level of a diabetes patient) of patients remotely with relative ease, even when the patients are not in a medical facility.

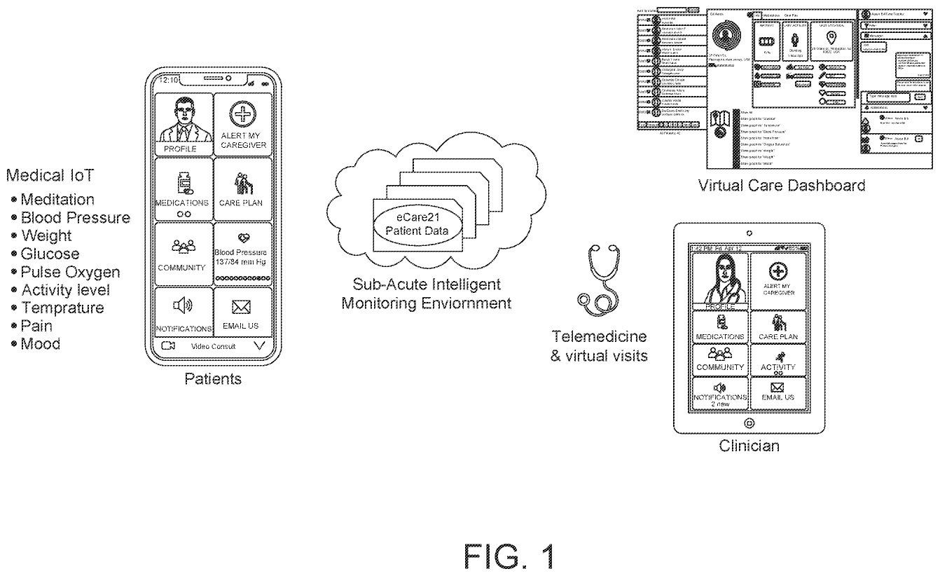

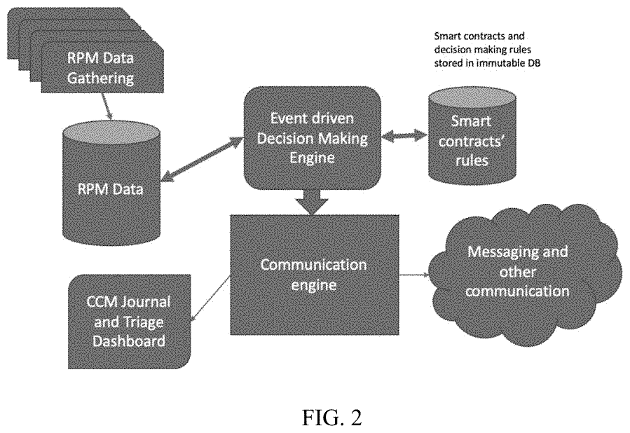

Embodiments of the subject invention support clinical decision-making by: collecting physiologic, medical, and/or psychometric data during the times when patients are outside of medical facilities; analyzing the data against smart contracts, medical data analytics, and/or historical data; and propagating actionable intelligence (see also, e.g., FIGS. 1 and 2). The actionable intelligence can be propagated to chronic care management systems (CCM) and/or electronic medical records management systems (ERMS), and delivered to family caregivers, homecare providers, care managers, hospital staff, and/or physicians in real time to improve results, decision making, and treatment protocols.

Embodiments of the present invention provide systems, methods, and user interfaces to access: care management services any time (e.g., 24 hours a day, seven days a week, 365 days a year (24/7)); continuity of care services; care management for chronic conditions, including medication management and assessment of the patient's medical, functional, and psychosocial needs; creation of a patient-centered care plan, with a written or electronic copy provided to patient; and management of care transitions, such as referrals or follow-up care after hospital or skilled nursing facility (SNF) discharge. This includes: transitional care management code; clinical summaries transmitted electronically (by HIPAA-compliant methods) to other providers; coordination with home and community-based clinical service providers, such as hospice; multiple ways for a patient and/or caregivers to contact providers, including via phone, a patient portal, and/or email; electronic capture and sharing of care plan information; a means of making electronic health records (EHR) and other patient records available 24/7 to all providers within the practice who may provide CCM services; and a means for making pertinent medical information available to providers outside the practice.

Software, processors, (non-transitory) machine-readable media (e.g., (non-transitory) computer-readable media), servers, computers, and/or network elements can be provided to integrate (e.g., with a rule-based engine) inputs from RPM devices (e.g., medical instruments used by patients outside a medical facility) and/or mobile applications with: hospital ERMS and compliant clinical documentation; Medicare CCM and RPM programs; MOMS; Medicare CPT Codes Support Systems; Senior Care Focus Systems; FQHCs and Visiting Physicians; Telehealth and Remote Case Management Systems; Medication Management Systems; Population Risk Management Systems; and/or Quality of Care, Treatment Protocols, and Cost Management Systems. This can all be done via, e.g., a cloud-based application that communicates with a remote server.

It is an objective of embodiments of the subject invention to provide analysis of physiologic, medical, and/or psychometric data to improve healthcare protocols, make better use of limited resources, extend limited resources or capabilities of highly-skilled practitioners, and reduce cost of care per patient. To achieve these and other objectives, embodiments provide methods and systems for confidentially and securely moving information between family caregivers, homecare providers, care managers, hospital staff, and physicians. Data inputs can be extracted from Food and Drug Administration (FDA) approved devices as well as standard over-the-counter health appliances, wireless devices, and/or simple monitoring tools able to track glucose, heart rate, physical activity, medication adherence, weight, calorie intake, and/or sleep among other key health indicators. The methods and systems can include family and/or caregivers in the process. All clinical and personal activity can be documented, and such documentation can be compliant with Medicare's new “Chronic Care Management” for integration with ERMS, benchmarking quality of care data-points against treatment protocols and cost in order to optimize care. Data analytics can be performed to improve decision making and optimization of in-house healthcare services, performance response, and patient engagement and self-care. Such data analytics can comprise, for example, machine learning (ML) and/or artificial intelligence (AI) (e.g., a support vector machine (SVM), a random decision forest (RDF), or a neural network such as a backpropagation neural network (BNN) or a recurrent neural network (RNN)). Robust, feature-rich, cloud-based monitoring and delivery of services can be provided, which allows for unlimited scaling and interoperability across diverse applications and platforms. Systems and methods can make use of an open architecture, technology-agnostic platform capable of integration with all RPM devices.

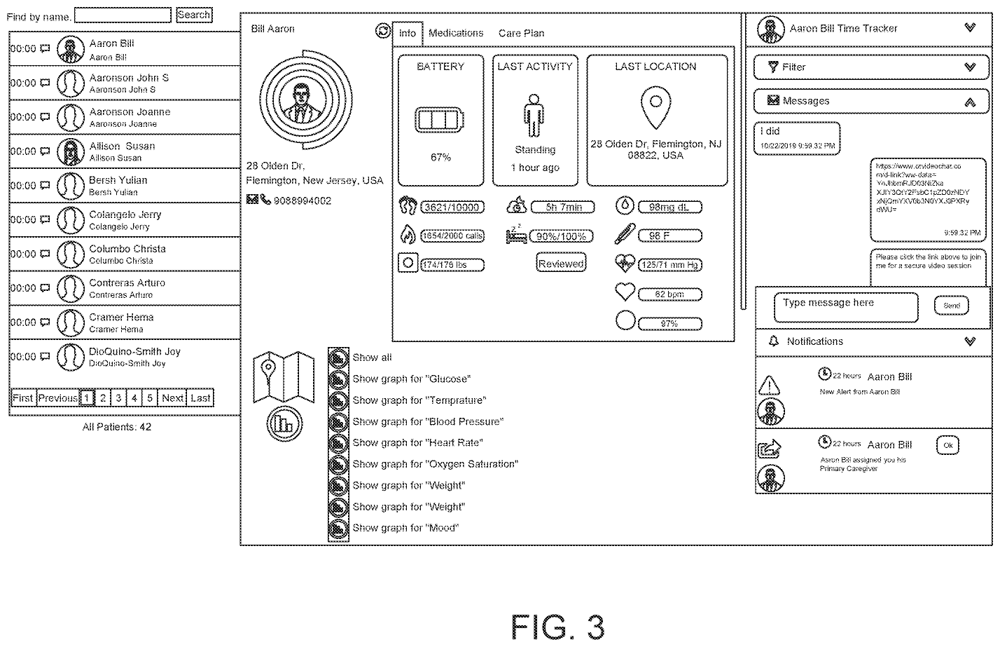

Systems and methods can receive data from various RPM devices and rationalize or categorize the data by source, based on the time the data was recorded and/or specificity of the data. The specificity of the data can be determined based on, for example, efficiency of the monitor (e.g., the system/method can determine from what RPM device the data came and how correct it is likely to be based on the level of accuracy of the source and when it was recorded). The data can be standardized such that data from any RPM device can be provided in a similar format, and the data can be integrated with other data received from the same and/or different RPM device(s) (e.g., based on time and relevancy). The standardized and/or integrated data can be provided to a database, which can be located, for example, on a remote server (e.g., a cloud-based server). The data can then be provided to records management systems of healthcare facilities, healthcare providers, and/or local caregivers (e.g., family caregivers), and this can be done in, for example, dashboard form (see, e.g., FIG. 3). Healthcare providers (and in some cases local caregivers) can act on the data.

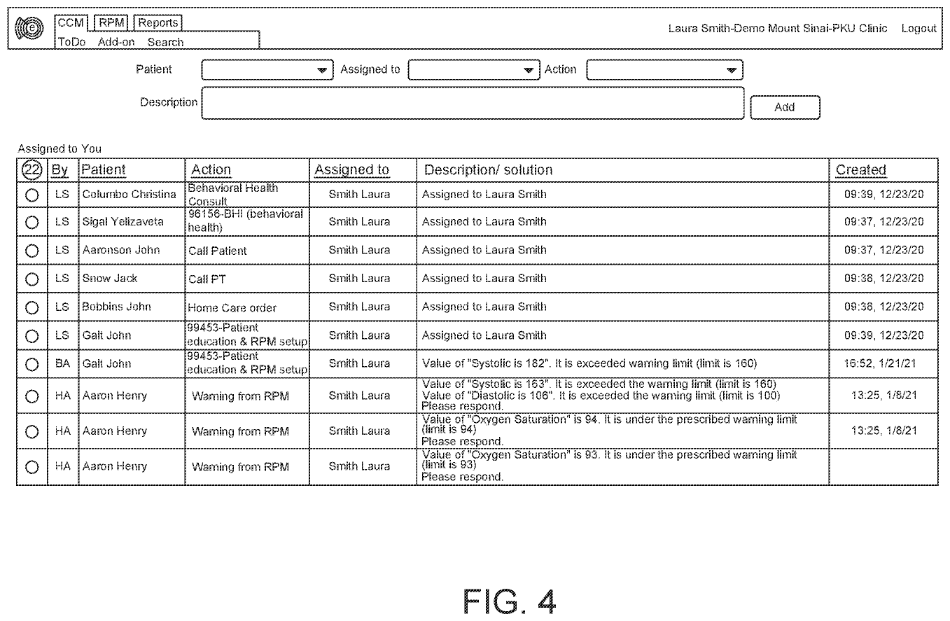

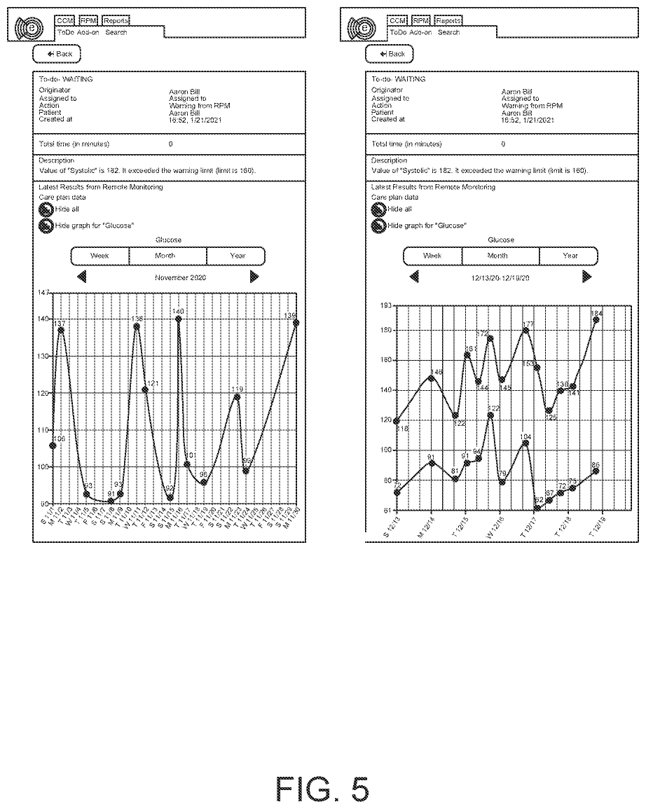

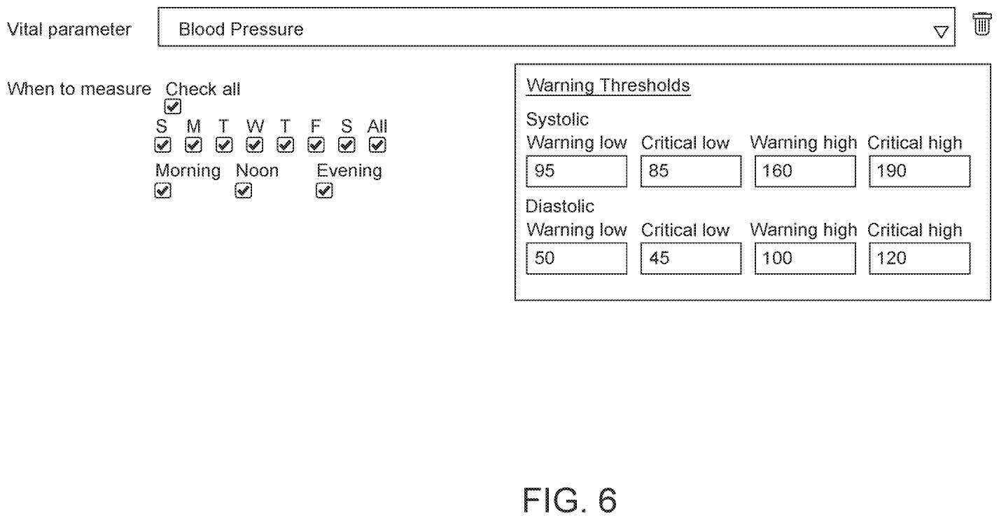

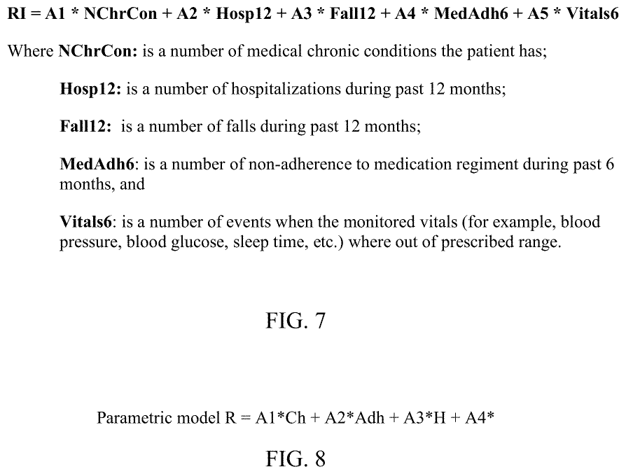

A healthcare provider can set at least one threshold for each RPM device of a patient (according to current FDA rules, this should only be a physician setting thresholds as it would qualify as medical advice). A first threshold can be set such that if a measured level of the RPM device exceeds or falls below the threshold (as may be relevant), a first alarm (see, e.g., FIGS. 4-8) is triggered such that this is brought to the attention of anyone viewing the dashboard data (e.g., a healthcare provider and/or local caregiver). The healthcare provider can then make a decision as to what action to take, if any, based on viewing all of the data on the dashboard. For example, the healthcare provider may determine that the patient should come in for a visit, either immediately, within a set period of time, or as convenient. A second threshold can be set such that if a measured level of the RPM device exceeds or falls below the threshold (as may be relevant), a second alarm (e.g., an emergency alarm) is triggered such that this is considered an urgent situation and is brought to the immediate attention of anyone viewing the dashboard data (e.g., a healthcare provider and/or local caregiver). This could require the patient to seek immediate medical care (e.g., by calling 911 or going to the hospital immediately). For example, a first threshold of 250 milligrams per deciliter (mg/dl) may be set for a patient with high cholesterol, and if the value is higher than this first threshold (but lower than the second threshold) a first alarm is triggered, and a second threshold of 350 mg/dl may be set, and if the measured value is higher than this second threshold a second alarm is triggered. The first and/or second thresholds can also be ranges instead of discrete values. For example, a first threshold range of 70-180 mg/dl may be set for blood sugar level of a diabetic patient, and a second threshold range of 60-250 mg/dl may be set; if the measured value is outside the first range but within the second range a first alarm is triggered, and if the measured value is outside the second range a second alarm is triggered. The values provided here are strictly for numerical exemplary purposes only and are not to be construed as actual medical advice for patients with the mentioned conditions.

Typically, health insurance companies or Medicare approves billing codes for a patient, and a provider (e.g., a physician) get a certain amount of compensation per patient, with a minimum amount of times the doctor must see the patient in a given period of time to qualify for the compensation. For example, a specialist may need to see a patient at least once a month to receive the regular per-patient compensation for that patient. This setup limits how many patients a provider can have, as the provider's time is limited. However, with systems and methods of embodiments of the subject invention, a provider can monitor a patient via the dashboard while the patient and family (and local caregivers) can see what is happening with RPM device measurements as well. Also, the provider is notified with an alarm and/or warning if thresholds set by the physician are passed; thus, the provider is able to monitor a higher number of patients, without substantially affecting level of care, and therefore can increase his or her total patient load. For example, a physician may be able to take on four times as many patients while using systems and methods of embodiments of the subject invention for dashboard monitoring, which would cut down on the number of in-person visits that are necessary.

In many embodiments, ML and/or AI can be used to improve a system or method over time. For example, ML and/or AI can be used to improve how data incoming from RPM devices is standardized, rationalized or categorized, and/or integrated. This can be done by comparing with past results and/or expected results. Examples of ML and/or AI that can be used include but are not limited to an SVM, an RDF, a BNN, or an RNN.

A smart contract can refer to an electronic protocol (e.g., a computer protocol) intended to digitally facilitate, verify, or enforce the negotiation or performance of a contract. In the medical field, care is paid for at different rates, and various groups create standards of care (e.g., treatment protocols) for various ailments. Systems and methods of embodiments of the subject invention increase value to patients and users (e.g., healthcare providers) by improving treatment protocols via monitoring information to potentially change treatment (e.g., dosing level, frequency of taking medication, etc.) over time to improve care. ML and/or AI can also be used for this, by monitoring the data from RPM devices over a period of time. The smart contracts can be stored on a database and can include rules about the threshold levels or ranges (as discussed herein) and alarms or alerts associated therewith.

In some embodiments, a “marketplace” type system/method can be provided, where remote care is supported and specific solutions are integrated therewith. For example, a database with data on pregnant women can be integrated with the system/method and provide more data for providers can treat a pregnant patient remotely without having to see the patient as often in person and without significantly affecting the standard of care. Additional specific solutions can be integrated over time, continuing to improve the data available to any provider(s) using the system/method.

Systems and methods of embodiments of the subject invention can be used with doctor cohorts (or similar groups). A doctor cohort combines records management of patients into a single records management system/database. Systems and methods can communicate with one or more doctor cohorts and/or one or more hospital records management system and/or one or more individual healthcare provider records management system, thereby increasing the data available and making any ML and/or AI used more effective at improving care and/or actionable intelligence.

As discussed in the Background, a consequence of age is that an ever increasing number of patients are requiring high-risk therapeutic treatment. The increased cost and demand for these services is rapidly exceeding the ability of providers to meet demand. Also, as a result of limited data and data collection healthcare providers are forced to overprescribe and over-treat to ensure minimum standards of care are met. Embodiments of the subject invention help address these problems.

Systems, methods, and apparatuses of embodiments of the subject invention provide the ability to capture data comparable to that which is captured in clinical observational studies, only the embodiments of the subject invention enable such capture on a 24/7 basis, resulting in better treatment protocols for any given provider, given healthcare facility, or given doctor cohort. Increased data capture and data analytics reduces the cost of managing chronic care and post-operative care by providing more comprehensive care, while limiting the scope of care provided to only that which is necessary, at much lower cost and risk. For example, current high-cost patients can receive the same or better care at home as compared to that which would be provided in a hospital or skilled nursing facilities.

Embodiments of the subject invention extend the resources and capabilities of hospitals and healthcare professionals into the patient's home at much lower costs. This allows highly skilled practitioners to treat more patients within any given time frame. Also, because of the cost savings, users (e.g., healthcare providers) can track a much larger set of vital statistics and health risk factors, allowing physicians to form a more complete view of the patient's health status and health risk factors facilitating proactive care.

Embodiments of the subject invention provide: a reduction in costs for Federally Qualified Healthcare Facilities (FQHF) so that they can serve a larger patient pool for the same resource pool; specialty care providers with a means for extending their practices to a wider patient-base while fostering a more healthy work-life-balance; and the ability to improve the care associated with, and cost of, high-risk therapeutic treatment programs (e.g., including but not limited to cardiac, vascular, nephrology, and/or pulmonary.)

Embodiments of the subject invention provide remote patient care capabilities that do not exist in the related art. Systems and methods provide commercial use of RPM for patients in the home or otherwise “at a distance” from a medical facility. Clinical decision making is supported and alerts are propagated based on the results of RPM and smart care contracts approved by the physician who prescribes the RPM. Alerts or alarms are propagated to mobile CCM systems or delivered to medical personnel in real time based on the severity of the patient's situation and the smart contract stored in the system, along with medical data analytics and/or historical data. Smart contracts can include but are not limited to blockchain and/or other immutable information storage means.

Systems and methods of embodiments of the subject invention can generate clean rules compliant, auditable clinical documentation and updates patients' care plans in real time. The systems and methods allow for homogenous use of wearable and in-home devices and human operator based data gathering. Using smart contracts can allow physicians and other medical personnel to make advanced decisions about a patient's treatment based on the real-time biometrical data.

Systems and methods can provide software, graphical user interfaces (GUIs), messaging, and the capability to integrate with RPM devices to capture data and perform CCM compliant with Current Procedural Terminology (CPT) rules, while also generating auditable, clinical documentation of Chronic Care Services provided. As of Jan. 1, 2015, Medicare began reimbursing for CCM services using CPT Code 99490. This service is for Medicare patients with multiple chronic conditions and is non-face-to-face. Since 2017, the Center for Medicare and Medicaid Services (CMS) has made a number of improvements to the program, including significantly increasing fees for CPT 99490 billing. The new reimbursements are in line with CMS' move to focus on higher quality primary care in an effort to reduce spending and improve outcomes.

In order to be eligible for CCM, patients must meet the following criteria: (a) a patient must have two or more chronic conditions; (b) the conditions are expected to last at least 12 months or until death of the patient; and (c) the conditions place the patient at significant risk of death, acute exacerbation (i.e., worsening of condition), decompensation (i.e., organ failure), or functional decline. CMS provides a summary of conditions that may apply to CCM; otherwise, the decision of what classifies as chronic is left up to the treating physician, along with the responsibility of providing detailed supporting chart documentation and an appropriate care plan. Physicians may bill for CCM services, and some non-physicians may as well, including but not limited to physician assistants, nurse practitioners, certified nurse midwives, and clinical nurse specialists.

CCM include eight basic elements: 1) access to care management services 24/7; 2) continuity of care; 3) Care management for chronic conditions, including medication management and assessment of the patient's medical, functional, and psychosocial needs; 4) creation of a patient-centered care plan, with a written or electronic copy provided to the patient; 5) management of care transitions, such as referrals or follow-up care after hospital or SNF discharge (this includes the transitional care management code, and clinical summaries must be transmitted electronically (by HIPAA-compliant methods) to other providers, with facsimiles not permitted); 6) coordination with home and community-based clinical service providers, such as hospice; 7) multiple ways for a patient and/or caregivers to contact providers, including via phone, the patient portal, or by email; and 8) electronic capture and sharing of care plan information. Providers must use a certified electronic health record (EHR), and the patient's records are to be available 24/7 to all providers within the practice who may provide CCM services. Providers outside the practice should be sent pertinent medical information electronically as well.

Core elements of CCM non-face-to-face clinical documentation include documenting that clinical staff spent at least 20 minutes of non-face-to-face time in a given month, recording the relevant information (e.g., date, time spent, name of provider, and the services provided), and billing Medicare using CPT code 99490. This should be billed only once per month per participating patient; in addition to billing 99490, the CPT codes for the chronic conditions should also be included, and the non-face-to-face time should never be rounded up.

Systems and methods of embodiments of the subject invention provide software, GUIs, messaging, and/or the capability to integrate with RPM devices to capture data. RPM devices use technology to allow the patient, a family healthcare provider, home healthcare professional, physician, and/or other healthcare providers to monitor disease, vital signs, and other physiologic, medical, and/or psychometric data without the need for the patient to have to go to a hospital or traditional clinical setting. Data inputs can be extracted from any FDA-approved device as well as standard over-the-counter health appliances, wireless devices, and/or simple monitoring tools able to track glucose, heart rate, physical activity, medication adherence, weight, calorie intake, and/or sleep apnea (among other key health indicators). Instruction(s) can also be provided to the patient via, e.g., audio, text-to-voice commands, graphics, video and/or text.

Systems and methods of embodiments of the subject invention provide software, GUIs, messaging, and/or the capability to integrate with Medical Office Management Systems (MOMS). MOMS include: software and devices to manage electronic health records and document patient care; medical billing to track activities and submit invoices to group healthcare plan administrators; and/or patient engagements by tracking schedule appointments, sending reminders for appointments and assisting with the management of follow up care. Physicians and staff can manage their patient population health through analysis of vital data. Embodiments extend and enhance MOMS by: tracking all healthcare services provided to the patient outside of a traditional clinical setting; tracking data associated with the patient's disease, vital signs and other physiologic, medical, and/or psychometric data; and/or organizing the data into actionable intelligence, sorted by priority.

Systems and methods of embodiments of the subject invention provide software, GUIs, and/or messaging to support RPM devices in accordance with Medicare requirements of CPT 99091. Within the 2018 Physician Fee Schedule, CMS provides physicians and other healthcare providers a new source of revenue for RPM billable under CPT 99091. The intent of the 2018 policy update was to offer compelling financial incentives for physicians to provide better care to patients, to improve care outcomes, and to lower the total cost of care. By using the appropriate technology and employing best practices, CPT 99091 can have a significant positive impact on the bottom line of the medical practice. CPT Code 99091 was created in 2002 for RPM; however, CMS has considered the work of the physician in reviewing and interpreting remote biometric data to be covered by management services codes already billed by the practice. In short, CPT 99091 was bundled with other clinical management services codes and could not be billed separately. Therefore, the code did little to promote the practical use of RPM. Under the new 2018 Physician Fee Schedule, incentives for RPM have dramatically improved with the unbundling of CPT 99490. As of Jan. 1, 2018, CPT 99091 has been unbundled and can be billed as a separate billable service. Under the 2018 Physician Fee Schedule, Medicare will pay $59 per patient per service period for RPM (with geographic fee adjustments). A growing number of commercial insurers also support reimbursement of CPT 99091 as well.

Systems and methods of embodiments of the subject invention provide software, GUIs, messaging, wireless and video capabilities, and/or the capability to provide CCM and RPM for senior patients (seniors). Senior care includes services that allow seniors to remain happy and independent in the comfort of their own home. Those services may include companionship, homemaker services, personal care, medication care and coordination, and advocacy.

With respect to companion care, embodiments can include a private social media application accessible on a permission-only basis from the internet or a telephone application to allow seniors and family care-providers to address the non-medical needs of the senior patient. The application can allow users to connect with friends, family members, and companions to provide assistance to seniors with everyday tasks, such as transportation to and from medical appointments, running errands, hobbies and other activities to stimulate mental awareness, and social interactions. It also provides scheduling, monitoring, communications and audit functions.

With respect to household services, embodiments can allow senior patient caregivers and family care providers to connect with those that can provide help around the house with such things as meal preparation, laundry, cleaning, and other household chores. Much like the other in-home services, seniors know they can rely on a friendly face to visit and take care of household tasks. Caregivers receive the same reliable training as for the other in-home services, and the services can be combined to assure that the senior is being taken care of according to their individual care plan. Geo-plotting, video, audio recording capabilities, support scheduling, monitoring, communications, and/or audit functions can be included in the sy stem/method.

With respect to personal care, embodiments can allow senior patient caregivers and family care providers to integrate the private social media application with the systems of third party certified nursing assistants (CNAs) who can provide assistance with CCM, grooming and personal care, activities of daily living, and/or medication management. Information including video, photographic images, and/or diagnostic tests can be stored and forwarded in support of Medicare reimbursement, and supervision and auditing of care providers can be provided.

With respect to medication administration, certified medication aides (CMAs) administer medications via various routes ensuring seniors are taking their medications according to doctor's orders. The service is especially beneficial to those with dementia or other diminished faculties. Embodiments of the subject invention can support CMAs connecting with senior patients, senior patient caregivers, and/or family care providers

Federally Qualified Healthcare Facilities (FQHCs) are community-based organizations that provide primary care and preventive care, including health, oral, and mental health/substance abuse services in underserved areas. System and methods of embodiments of the subject invention provide software, GUIs, messaging, and/or the capability to provide CCM, RPM, clinical documentation and Medicare support systems, MOMS, Telehealth, and/or Remote Case Management Systems that can assist with FQHCs.

Telehealth encompasses elements of telemedicine, but it also includes administrative tasks, remote patient monitoring, and other non-direct interactions. Live or synchronous interactions between a patient and provider or two providers can be used to extend limited resources and to cover a larger service area in less time and more efficiently. Store-and-forward capabilities like video, photographic images, and diagnostic tests allow a primary care provider or specialist view them at a later date. Systems and methods can support this.

Telemedicine typically refers specifically to live video consults. System and methods of embodiments of the subject invention provide software, GUIs, messaging, wireless, and/or video capabilities to allow a doctor to consult directly with a patient or another physician securely, in real time from anywhere where there is a wireless signal and/or internet connection.

Medication management refers to a strategy for engaging with patients and caregivers to create a complete and accurate medication list as well as ensuring that the patient is taking the prescribed medication, in the required amounts and the required intervals. System and methods of embodiments of the subject invention provide software, GUIs, messaging, wireless capabilities, video capabilities, scheduling capabilities, and/or a private social media application to allow family care providers, CMAs, and/or visiting nurses to assist with and oversee medication management.

Population risk management generally refers to the ability to severity-adjust risk stratification of a population group. System and methods of embodiments of the subject invention provide software, GUIs, messaging, and/or the capability to integrate with RPM devices to capture physiologic, medical, and/or psychometric data. Systems and methods can integrate with ERMS systems to capture data against a rules based engine to create actionable intelligence that can be used to support early intervention programs, manage health risk factors, and/or promote health and wellness habits.

The National Institute of Health (NIH) defines benchmarking in health care as a process of comparative evaluation and identification of the underlying causes leading to high levels of performance. Health care benchmarking allows FQHCs and physician groups (or physician cohorts) to track performance for a given outcome and apply specific clinical practices that are the most effective. They may also track structural, cultural, or organizational features that contribute to excellent outcomes. System and methods of embodiments of the subject invention provide software, GUIs, messaging, and/or the capability to integrate with RPM devices to capture a wider range of physiologic, medical, and/or psychometric data that can be used to benchmark quality of care. Systems and methods can also track all healthcare-related activities of healthcare providers and physicians. Tools can be provided to analyze data, and a rules engine can be provided for physicians to better control and track treatment protocols.

Embodiments of the subject invention provide systems, methods, and apparatuses for collecting a patient's RPM data and integrating said RPM data with a healthcare facility's ERMS. Embodiments of the subject invention provide systems, methods, and apparatuses for capturing all healthcare-related activities associated with CCM and/or RPM and submitting said health-related activities to Medicare for reimbursement. Embodiments of the subject invention provide systems, methods, and apparatuses for analyzing a patient's RPM data based on pre-recorded smart contract rules and notifying clinical personnel about medical needs of the patient. Embodiments of the subject invention provide systems, methods, and apparatuses for allowing medical staff (e.g., doctors, nurses) to create smart contracts for analysis of RPM data. Embodiments of the subject invention provide systems, methods, and apparatuses for storing smart contracts in an internal or external data warehouse including, but not limited to, in a blockchain format. Embodiments of the subject invention provide systems, methods, and apparatuses for supporting clean and compliant collection, auditing, and storage of HIPAA compliant clinical documentation associated with CCM and RPM.

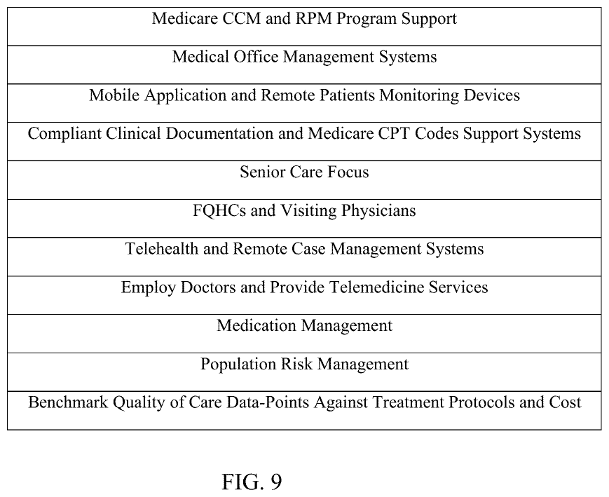

Systems and embodiments of the subject invention provide numerous advantages over related art systems and methods (see also, e.g., FIG. 9). One advantage is the ability for healthcare providers to capture a patient's physiologic, medical, and/or psychological status 24 hours a day, 7 days a week, 365 days a year, particularly outside of a healthcare facility. Another advantage is realized by allowing the data that is captured to be integrated into the healthcare facility's ERMS in real time. Yet another advantage is attained by analyzing the physiologic, medical, and/or psychometric data captured against a rules-based engine (e.g., including rules about triggering alarms based on exceeding or dropping below certain threshold values and/or registering outside of threshold ranges) to create actionable intelligence (such analysis can be accomplished via, e.g., ML and/or AI). Still yet another advantage is attained by analyzing the physiologic, medical, and/or psychometric data captured against smart contracts, medical data analytics, and/or historical data to create actionable intelligence (such analysis can be accomplished via, e.g., ML and/or AI). A still further advantage is realized by transmitting the actionable intelligence to the person or persons best able to act on the information at the lowest cost and/or highest level of care. A further advantage is attained by transmitting the actionable intelligence to family caregivers, homecare providers, case managers, hospital staff, and/or physicians in real time in order to reduce cost and/or improve care. Another advantage is realized by analyzing physiologic, medical, and/or psychometric data to recommend improved treatment protocols for patients, which may include a reduction in scope, medication, or treatment protocols, an improvement in treatment protocols, or some combination of all of these (such analysis can be accomplished via, e.g., ML and/or AI). Another advantage is realized by analyzing physiologic, medical, and/or psychometric data of many patients over time to conduct longitudinal studies based on age, medical condition, and/or certain physiologic, medical, and/or psychometric parameters common to a cohort of patients (such analysis can be accomplished via, e.g., ML and/or AI). These and other advantages can be realized by systems and methods for providing CCM and using mobile applications and devices for RPM, along with the integration of RPM results into CCM (e.g., using smart contracts and/or other electronic decision making instruments based on the physician's prescription, various standards of care, medical guidelines, and/or group healthcare plan guidelines). Systems and methods can provide improved understanding of digital health, preventive care, senior care, care coordination, CCM, RPM, and/or post-surgical care.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

The subject invention includes, but is not limited to, the following exemplified embodiments.

Embodiment 1. A system for remote monitoring of at least one medical patient, the system comprising:

a processor;

a first display; and

a (non-transitory) machine-readable medium in operable communication with the processor, the machine-readable medium having instructions stored thereon that, when executed by the processor, perform the following steps:
collecting remote patient monitoring (RPM) data from at least one RPM device in use by the at least one medical patient outside of a medical facility, the collected RPM data comprising measurement values of the at least one RPM device;
standardizing the collected RPM data;
categorizing the standardized RPM data based on a time the RPM data was collected from the at least RPM device and the type of RPM device from which the RPM data was collected;
storing the categorized RPM data in a database;
(optionally providing the categorized RPM data to an electronic records management system (ERMS) of the medical facility); and
organizing the categorized RPM data in a dashboard in a graphical user interface (GUI) displayed on the first display where it is monitored by a medical professional and used as actionable intelligence in providing medical care to the at least one medical patient.

Embodiment 2. The system according to embodiment 1, further comprising a memory in operable communication with the processor and the machine-readable medium.

Embodiment 3. The system according to any of embodiments 1-2, the database comprising at least one smart contract with a first rule regarding RPM data collected from a first RPM device of the at least one RPM device and a second rule regarding RPM data collected from the first RPM device of the at least one RPM device,

wherein the first rule requires a first alarm is triggered on the dashboard if a measurement value of the first RPM device exceeds or falls below a first threshold or is outside of a first threshold range, and

wherein the second rule requires a second alarm is triggered on the dashboard if the measurement value of the first RPM device exceeds or falls below a second threshold (which can be greater than or less than the first threshold if the second alarm is triggered by exceeding or falling below, respectively, the second threshold) or is outside of a second threshold range (with upper and lower bounds that are greater and less, respectively, than those of the first threshold), the second alarm being an indicator of an emergency situation for the at least one medical patient.

Embodiment 4. The system according to embodiment 3, the database storing the at least one smart contract in a blockchain format.

Embodiment 5. The system according to any of embodiments 1-4, the database being stored on a remote server with which the processor communicates.

Embodiment 6. The system according to embodiment 5, wherein the remote server is a cloud-based server.

Embodiment 7. The system according to any of embodiments 1-6, wherein the instructions when executed further perform the step of providing the categorized RPM data to a local caregiver of the at least one medical patient, and

wherein the dashboard is displayed on a second display where it is monitored by the local caregiver.

Embodiment 8. The system according to any of embodiments 1-7, wherein the instructions when executed further perform the step of submitting the collected RPM data to a Medicare server for Medicare reimbursement related to the at least one medical patient, wherein the Medicare reimbursement is provided to the medical professional, the medical facility, or both.

Embodiment 9. The system according to any of embodiments 1-8, the instructions when executed further perform the step of utilizing machine learning (ML), artificial intelligence (AI), or both to improve the steps of standardizing and categorizing.

Embodiment 10. The system according to any of embodiments 1-9, the instructions when executed further perform the step of utilizing machine learning (ML), artificial intelligence (AI), or both to improve treatment protocols by monitoring over a period of time the categorized RPM data.

Embodiment 11. The system according to any of embodiments 1-10, the instructions when executed further perform the steps of:

collecting cohort data from at least one doctor cohort;

standardizing the collected cohort data;

categorizing the standardized cohort data;

storing the categorized cohort data in the database; (optionally providing the categorized cohort data to the ERMS of the medical facility); and

organizing the categorized cohort data in the dashboard in the GUI displayed on the first display where it is monitored by the medical professional and used as actionable intelligence in providing medical care.

Embodiment 12. The system according to embodiment 11, the instructions when executed further perform the step of utilizing machine learning (ML), artificial intelligence (AI), or both to improve treatment protocols by monitoring over a period of time the categorized RPM data and the categorized cohort data.

Embodiment 13. A method for remote monitoring of at least one medical patient, the method comprising:

collecting (e.g., by a processor) remote patient monitoring (RPM) data from at least one RPM device in use by the at least one medical patient outside of a medical facility, the collected RPM data comprising measurement values of the at least one RPM device;

standardizing (e.g., by the processor) the collected RPM data;

categorizing (e.g., by the processor) the standardized RPM data based on a time the RPM data was collected from the at least RPM device and the type of RPM device from which the RPM data was collected;

storing (e.g., by the processor) categorized RPM data in a database;

(optionally providing (e.g., by the processor) the categorized RPM data to an electronic records management system (ERMS) of the medical facility); and

organizing (e.g., by the processor) the categorized RPM data in a dashboard in a graphical user interface (GUI) displayed on a first display where it is monitored by a medical professional and used as actionable intelligence in providing medical care to the at least one medical patient.

Embodiment 14. The method according to embodiment 13, the database comprising at least one smart contract with a first rule regarding RPM data collected from a first RPM device of the at least one RPM device and a second rule regarding RPM data collected from the first RPM device of the at least one RPM device,

wherein the first rule requires a first alarm is triggered on the dashboard if a measurement value of the first RPM device exceeds or falls below a first threshold or is outside of a first threshold range, and

wherein the second rule requires a second alarm is triggered on the dashboard if the measurement value of the first RPM device exceeds or falls below a second threshold (which can be greater than or less than the first threshold if the second alarm is triggered by exceeding or falling below, respectively, the second threshold) or is outside of a second threshold range (with upper and lower bounds that are greater and less, respectively, than those of the first threshold), the second alarm being an indicator of an emergency situation for the at least one medical patient.

Embodiment 15. The method according to embodiment 14, the database storing the at least one smart contract in a blockchain format.

Embodiment 16. The method according to any of embodiments 13-15, the database being stored on a remote server with which the processor communicates.

Embodiment 17. The method according to embodiment 16, wherein the remote server is a cloud-based server.

Embodiment 18. The method according to any of embodiments 13-17, wherein the instructions when executed further perform the step of providing the categorized RPM data to a local caregiver of the at least one medical patient, and

wherein the dashboard is displayed on a second display where it is monitored by the local caregiver.

Embodiment 19. The method according to any of embodiments 13-18, wherein the instructions when executed further perform the step of submitting the collected RPM data to a Medicare server for Medicare reimbursement related to the at least one medical patient, wherein the Medicare reimbursement is provided to the medical professional, the medical facility, or both.

Embodiment 20. The method according to any of embodiments 13-19, the instructions when executed further perform the step of utilizing machine learning (ML), artificial intelligence (AI), or both to improve the steps of standardizing and categorizing.

Embodiment 21. The method according to any of embodiments 13-20, the instructions when executed further perform the step of utilizing ML, AI, or both to improve treatment protocols by monitoring over a period of time the categorized RPM data.

Embodiment 22. The method according to any of embodiments 13-21, the instructions when executed further perform the steps of:

collecting cohort data from at least one doctor cohort;

standardizing the collected cohort data;

categorizing the standardized cohort data;

storing the categorized cohort data in the database; (optionally providing the categorized cohort data to the ERMS of the medical facility); and

organizing the categorized cohort data in the dashboard in the GUI displayed on the first display where it is monitored by the medical professional and used as actionable intelligence in providing medical care.

Embodiment 23. The method according to embodiment 22, the instructions when executed further perform the step of utilizing machine learning (ML), artificial intelligence (AI), or both to improve treatment protocols by monitoring over a period of time the categorized RPM data and the categorized cohort data.

Embodiment 24. The system according to any of embodiments 9, 10, or 12, or the method according to any of embodiments 20, 21, or 23 wherein the ML, AI, or both comprises a support vector machine (SVM), a random decision forest (RDF), or a neural network (e.g., a backpropagation neural network (BNN) or a recurrent neural network (RNN)).

Embodiment 25. The system according to any of embodiments 1-11 or 24, or the method according to any of embodiments 12-24 wherein the categorized RMS data (and categorized cohort data, where applicable) is provided to an ERMS of the medical professional (which may be separate from the ERMS of the medical facility).

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

What is claimed is:

1. A system for remote monitoring of at least one medical patient, the system comprising:
a processor;
a first display; and
a machine-readable medium in operable communication with the processor, the machine-readable medium having instructions stored thereon that, when executed by the processor, perform the following steps:
collecting remote patient monitoring (RPM) data from at least one RPM device in use by the at least one medical patient outside of a medical facility, the collected RPM data comprising measurement values of the at least one RPM device;
standardizing the collected RPM data;
categorizing the standardized RPM data based on a time the RPM data was collected from the at least RPM device and the type of RPM device from which the RPM data was collected;
storing the categorized RPM data in a database; and
organizing the categorized RPM data in a dashboard in a graphical user interface (GUI) displayed on the first display where it is monitored by a medical professional and used as actionable intelligence in providing medical care to the at least one medical patient.

2. The system according to claim 1, the database comprising at least one smart contract with a first rule regarding RPM data collected from a first RPM device of the at least one RPM device and a second rule regarding RPM data collected from the first RPM device of the at least one RPM device,
wherein the first rule requires a first alarm is triggered on the dashboard if a measurement value of the first RPM device exceeds or falls below a first threshold or is outside of a first threshold range, and
wherein the second rule requires a second alarm is triggered on the dashboard if the measurement value of the first RPM device exceeds or falls below a second threshold or is outside of a second threshold range, the second alarm being an indicator of an emergency situation for the at least one medical patient.

3. The system according to claim 2, the database storing the at least one smart contract in a blockchain format.

4. The system according to claim 1, the database being stored on a remote server with which the processor communicates,
wherein the remote server is a cloud-based server.

5. The system according to claim 1, wherein the instructions when executed further perform the step of providing the categorized RPM data to an electronic records management system (ERMS) of the medical facility.

6. The system according to claim 1, wherein the instructions when executed further perform the step of providing the categorized RPM data to a local caregiver of the at least one medical patient, and
wherein the dashboard is displayed on a second display where it is monitored by the local caregiver.

7. The system according to claim 1, wherein the instructions when executed further perform the step of submitting the collected RPM data to a Medicare server for Medicare reimbursement related to the at least one medical patient, wherein the Medicare reimbursement is provided to the medical professional, the medical facility, or both.

8. The system according to claim 1, the instructions when executed further perform the step of utilizing machine learning (ML), artificial intelligence (AI), or both to improve the steps of standardizing and categorizing.

9. The system according to claim 1, the instructions when executed further perform the step of utilizing machine learning (ML), artificial intelligence (AI), or both to improve treatment protocols by monitoring over a period of time the categorized RPM data.

10. The system according to claim 1, the instructions when executed further perform the steps of:
collecting cohort data from at least one doctor cohort;
standardizing the collected cohort data;
categorizing the standardized cohort data;
storing the categorized cohort data in the database; and
organizing the categorized cohort data in the dashboard in the GUI displayed on the first display where it is monitored by the medical professional and used as actionable intelligence in providing medical care.

11. The system according to claim 10, the instructions when executed further perform the step of utilizing machine learning (ML), artificial intelligence (AI), or both to improve treatment protocols by monitoring over a period of time the categorized RPM data and the categorized cohort data.

12. A method for remote monitoring of at least one medical patient, the method comprising:
collecting remote patient monitoring (RPM) data from at least one RPM device in use by the at least one medical patient outside of a medical facility, the collected RPM data comprising measurement values of the at least one RPM device;
standardizing the collected RPM data;
categorizing the standardized RPM data based on a time the RPM data was collected from the at least RPM device and the type of RPM device from which the RPM data was collected;
storing the categorized RPM data in a database; and
organizing the categorized RPM data in a dashboard in a graphical user interface (GUI) displayed on a first display where it is monitored by a medical professional and used as actionable intelligence in providing medical care to the at least one medical patient.

13. The method according to claim 12, the database comprising at least one smart contract with a first rule regarding RPM data collected from a first RPM device of the at least one RPM device and a second rule regarding RPM data collected from the first RPM device of the at least one RPM device,
wherein the first rule requires a first alarm is triggered on the dashboard if a measurement value of the first RPM device exceeds or falls below a first threshold or is outside of a first threshold range, and
wherein the second rule requires a second alarm is triggered on the dashboard if the measurement value of the first RPM device exceeds or falls below a second threshold or is outside of a second threshold range, the second alarm being an indicator of an emergency situation for the at least one medical patient.

14. The method according to claim 13, the database storing the at least one smart contract in a blockchain format.

15. The method according to claim 12, the database being stored on a remote server with which the processor communicates, and
wherein the remote server is a cloud-based server.

16. The method according to claim 12, wherein the instructions when executed further perform the step of providing the categorized RPM data to a local caregiver of the at least one medical patient, and
wherein the dashboard is displayed on a second display where it is monitored by the local caregiver.

17. The method according to claim 12, wherein the instructions when executed further perform the step of submitting the collected RPM data to a Medicare server for Medicare reimbursement related to the at least one medical patient, wherein the Medicare reimbursement is provided to the medical professional, the medical facility, or both.

18. The method according to claim 12, the instructions when executed further perform the step of:
a) utilizing machine learning (ML), artificial intelligence (AI), or both to improve the steps of standardizing and categorizing;
b) utilizing ML, AI, or both to improve treatment protocols by monitoring over a period of time the categorized RPM data; or
c) both a) and b).

19. The method according to claim 12, the instructions when executed further perform the steps of:
collecting cohort data from at least one doctor cohort;
standardizing the collected cohort data;
categorizing the standardized cohort data;
storing the categorized cohort data in the database; and
organizing the categorized cohort data in the dashboard in the GUI displayed on the first display where it is monitored by the medical professional and used as actionable intelligence in providing medical care.

20. The method according to claim 19, the instructions when executed further perform the step of utilizing machine learning (ML), artificial intelligence (AI), or both to improve treatment protocols by monitoring over a period of time the categorized RPM data and the categorized cohort data.
